(19) United States
(12) Patent Application Publication
(10) Pub. No.: US 2022/0117044 A1
(43) Pub. Date: Apr. 14, 2022

(54) MICROHEATER, GAS SENSOR, AND METHOD FOR MANUFACTURING MICROHEATER

(71) Applicant: ROHM CO., LTD., Kyoto (JP)

(72) Inventors: Shunsuke AKASAKA, Kyoto (JP); Encho BOKU, Kyoto (JP); Hiroyuki YUJI, Kyoto (JP)

(73) Assignee: ROHM CO., LTD., Kyoto (JP)

(21) Appl. No.: 17/561,912

(22) Filed: Dec. 24, 2021

Related U.S. Application Data
(63) Continuation of application No. PCT/JP2020/018143, filed on Apr. 28, 2020.

(30) Foreign Application Priority Data
Jun. 27, 2019 (JP) 2019-119994

(51) Int. Cl. H05B 3/28; C23C 14/08; G01N 33/00; C23C 28/04; C23C 14/34; C23C 16/34; G01K 7/18; C23C 16/40

(52) U.S. Cl. CPC H05B 3/28; C23C 14/083; G01N 33/0027; C23C 28/042; H05B 2203/017; C23C 16/345; G01K 7/186; C23C 16/401; C23C 14/34

(57) ABSTRACT

A microheater includes a first insulating layer, a first adhesion layer on the first insulating layer, a wiring layer on the first adhesion layer, a second adhesion layer that covers the wiring layer, and a second insulating layer above the first insulating layer and on the second adhesion layer. In the microheater, the wiring layer contains platinum, the first adhesion layer and the second adhesion layer each contain a metal oxide, and the metal oxide has an oxygen-deficient region in which the oxygen is deficient in the stoichiometric ratio of metal to oxygen.

### CROSS REFERENCE TO RELATED APPLICATIONS AND INCORPORATION BY REFERENCE

This is a continuation application (CA) of PCT Application No. PCT/JP2020/018143, filed on Apr. 28, 2020, which claims priority to Japan Patent Application No. P2019-119994 filed on Jun. 27, 2019 and is based upon and claims the benefit of priority from prior Japanese Patent Application No. P2019-119994 filed on Jun. 27, 2019 and PCT Application No. PCT/JP2020/018143, filed on Apr. 28, 2020; the entire contents of each of which are incorporated herein by reference.

### TECHNICAL FIELD

The present embodiment relates to a microheater, a gas sensor, and a method for manufacturing a microheater.

### BACKGROUND OF THE INVENTION

A microheater is used in various devices such as gas sensors and humidity sensors, for example. A gas sensor has a microheater, a temperature sensor, and the like. Such a microheater uses platinum to generate heat, and for example, a microheater that has a platinum film formed in a zigzag shape is disclosed.

In addition, a wiring portion of a microheater which is made of platinum is designed to be normally used at about 300 to 400° C. The wiring portion is generally covered with a refractory metal, which is the material of an adhesion layer, or a nitride thereof.

However, if a microheater is used in a high temperature region (for example, about 500° C.) at or above the abovementioned temperature range, the nitride reacts with platinum due to heating, and a void occurs in a wiring portion. The void gradually expands due to being continuously heated, eventually the wiring portion is disconnected, and there is a possibility of causing an operation failure of the microheater.

In order to suppress disconnections such as the above, consideration has been given to use an oxide insulating layer which does not react with platinum in a high temperature region instead of using a nitride layer. However, the adhesion between platinum and an oxide is insufficient, and film separation is likely to occur. Therefore, it has been difficult to make an oxide insulating layer function as an adhesion layer.

### SUMMARY OF THE INVENTION

The present embodiment can provide a microheater with an adhesion layer that suppresses the occurrence of a void in a wiring portion and ensures adhesion with a wiring portion. Another embodiment can provide a gas sensor that includes the microheater. Still another embodiment can provide a method for manufacturing the microheater.

### BRIEF DESCRIPTION OF THE DRAWINGS

FIG. 1 is a schematic plan view showing a structure of a microheater of an aspect of the present embodiment.

FIGS. 2A to 2C are schematic cross sectional views showing a structure of a microheater of an aspect of the present embodiment.

FIG. 3 is a schematic cross sectional view for explaining a method for manufacturing a microheater of an aspect of the present embodiment, and is also a process diagram showing that an insulating layer 12, a nitride layer 14, and an insulating layer 16 are sequentially formed in this order on a substrate 10.

FIG. 4 is a schematic cross sectional view for explaining a method for manufacturing a microheater of an aspect of the present embodiment, and is also a process diagram showing that an adhesion layer 18a, a wiring layer 18b, and an adhesion layer 18c1 are sequentially formed in this order.

FIG. 5 is a schematic cross sectional view for explaining a method for manufacturing a microheater of an aspect of the present embodiment, and is also a process diagram showing the removal of a part of an adhesion layer 18c1.

FIG. 6 is a schematic cross sectional view for explaining a method for manufacturing a microheater of an aspect of the present embodiment, and is also a process diagram showing the removal of a part of a wiring layer 18b.

FIG. 7 is a schematic cross sectional view for explaining a method for manufacturing a microheater of an aspect of the present embodiment, and is also a process diagram showing the formation of an adhesion layer 18c2.

FIG. 8 is a schematic cross sectional view for explaining a method for manufacturing a microheater of an aspect of the present embodiment, and is also a process diagram showing the formation of an adhesion layer 18c.

FIG. 9 is a schematic cross sectional view for explaining a method for manufacturing a microheater of an aspect of the present embodiment, and is also a process diagram showing that an insulating layer 20 and a nitride layer 22 are sequentially formed in this order.

FIG. 10 is a schematic cross sectional view for explaining a method for manufacturing a microheater of an aspect of the present embodiment, and is also a process diagram showing the formation of a temperature sensor 24.

FIG. 11 is a schematic cross sectional view for explaining a method for manufacturing a microheater of an aspect of the present embodiment, and is also a process diagram showing the formation of an opening which is for connecting a pair of electrodes disposed outside of a microheater with a wiring layer 18b.

FIG. 12 is a schematic cross sectional views for explaining a method for manufacturing a microheater of an aspect of the present embodiment, and is also a process diagram showing the formation of an opening which extends and reaches a substrate 10.

FIG. 13 is a schematic cross sectional view for explaining a method for manufacturing a microheater of an aspect of the present embodiment, and is also a process diagram showing the removal of a part of a substrate 10 by means of etching or the like.

FIG. 14 is a schematic plan view of a gas sensor having a microheater of an aspect of the present embodiment.

FIG. 15 is a schematic cross sectional view of a gas sensor having a microheater of an aspect of the present embodiment.

FIG. 16 shows an image of a cross section TEM of a microheater of an example.

FIG. 17A shows images of a cross section TEM of a microheater of an example, and shows a cross section image in which an adhesion layer 18a and an adhesion layer 18c of a heater layer 18 are titanium oxide layers (TiO_{1.1}).

FIG. 17B shows images of a cross section TEM of a microheater of an example, and shows a cross section image in which an adhesion layer 18a and an adhesion layer 18c are titanium oxide layers (TiO₂).

FIG. 17C shows images of a cross section TEM of a microheater of an example, and shows a cross section image in which an adhesion layer 18a and an adhesion layer 18c are titanium nitride layers (TiN).

FIG. 18A shows surface micrographs of a microheater of an example, and shows a surface image in which an adhesion layer 18a and an adhesion layer 18c of a heater layer 18 are titanium oxide layers (TiO_{0.9}).

FIG. 18B shows surface micrographs of a microheater of an example, and shows a surface image in which an adhesion layer 18a and an adhesion layer 18c are titanium oxide layers (TiO_{1.1}).

FIG. 18C shows surface micrographs of a microheater of an example, and shows a surface image in which an adhesion layer 18a and an adhesion layer 18c are titanium oxide layers (TiO_{1.4}).

FIG. 19A shows surface micrographs of a microheater of an example, and shows a surface image in which an adhesion layer 18a and an adhesion layer 18c of a heater layer 18 are titanium nitride layers (TiN).

FIG. 19B shows surface micrographs of a microheater of an example, and shows a surface image in which an adhesion layer 18a and an adhesion layer 18c are titanium oxide layers (TiO_{0.5}).

FIG. 19C shows surface micrographs of a microheater of an example, and shows a surface image in which an adhesion layer 18a and an adhesion layer 18c are titanium oxide layers (TiO₂).

FIG. 20 is a diagram showing evaluation results of the temperature of a membrane and electric power applied to a wiring layer 18b in a microheater of an example.

FIG. 21 is a diagram showing evaluation results of a cycle characteristic of a microheater of an example.

### DETAILED DESCRIPTION OF THE INVENTION

Next, the present embodiment will be described with reference to the drawings. In the drawings described below, the same or similar parts are denoted by the same or similar reference numerals. It should be noted, however, that the drawings are schematic, and that the relationships or the like between the thickness and the planar dimensions of each component are different from those in reality. Therefore, the specific thickness and dimensions should be determined in consideration of the following description. In addition, it is needless to say that the drawings include portions in which the relationships and the ratios of the dimensions are different from each other.

Further, the following embodiments exemplify an apparatus and a method for embodying a technical idea, and do not specify the material, shape, structure, disposition, and the like of each component. In the present embodiment, various modifications can be made within the scope of claims.

One aspect of the present embodiment is as follows.

[1] A microheater comprising: a first insulating layer; a first adhesion layer on the first insulating layer; a wiring layer on the first adhesion layer; a second adhesion layer that covers the wiring layer; and a second insulating layer above the first insulating layer and on the second adhesion layer, wherein the wiring layer includes platinum, the first adhesion layer and the second adhesion layer each include a metal oxide, and the metal oxide includes an oxygen-deficient region in which oxygen is deficient in a stoichiometric ratio of metal to oxygen.

[2] The microheater according to [1], wherein the oxygen in the oxygen-deficient region is 30 to 80% of oxygen of a stoichiometric composition of the metal oxide, and the metal includes one selected from the group consisting of titanium, chromium, tungsten, molybdenum, and tantalum.

[3] The microheater according to [1] or [2], wherein the metal is titanium, and a stoichiometric ratio of metal to oxygen of the metal oxide is more than 1:0.5 and 1:1.5 or less.

[4] The microheater according to any one of [1] to [3], wherein the oxygen-deficient region includes a region in which the amount of oxygen gradually increases from an interface between the wiring layer and the first adhesion layer toward the first insulating layer, and a region in which the amount of oxygen gradually increases from an interface between the wiring layer and the second adhesion layer toward the second insulating layer.

[5] The microheater according to any one of [1] to [4], further comprising: a temperature sensor on the second insulating layer, wherein the wiring layer includes platinum, the second insulating layer includes an oxide insulating layer and a nitride layer on the oxide insulating layer, the wiring layer is connected to each of a pair of electrodes and includes a first bellows structure, the temperature sensor includes a second bellows structure, an angle formed between a straight line portion of the first bellows structure and a straight line portion of the second bellows structure is in a range from 45 degrees to 135 degrees, and the temperature sensor includes a metal oxide layer and a metal layer on the metal oxide layer.

[6] A gas sensor that includes the microheater according to [5], wherein a metal oxide in the metal oxide layer includes the oxygen-deficient region in which the oxygen is deficient in a stoichiometric ratio of metal to oxygen, and the metal oxide in the metal oxide layer includes a material which is the same as that of the metal oxide in the first adhesion layer and the second adhesion layer.

[7] A method for manufacturing a microheater comprising: forming a first insulating layer; forming a first adhesion layer on the first insulating layer; forming a wiring layer on the first adhesion layer; forming a second adhesion layer that covers a side surface of the wiring layer on the wiring layer; and forming a second insulating layer above the first insulating layer and on the second adhesion layer, wherein the first adhesion layer and the second adhesion layer each include a metal oxide, and the metal oxide includes an oxygen-deficient region in which oxygen is deficient in a stoichiometric ratio of metal to oxygen.

[8] The method for manufacturing the microheater according to [7], wherein the oxygen in the oxygen-deficient region is 30 to 80% of oxygen of a stoichiometric composition of the metal oxide, and the metal includes one selected from the group consisting of titanium, chromium, tungsten, molybdenum, and tantalum.

[9] The method for manufacturing the microheater according to [7] or [8], wherein the metal is titanium, and a stoichiometric ratio of metal to oxygen of the metal oxide is more than 1:0.5 and 1:1.5 or less.

[10] The method for manufacturing the microheater according to any one of [7] to [9], wherein the oxygen-deficient region includes a region in which the amount of oxygen gradually increases from an interface between the wiring layer and the first adhesion layer toward the first insulating layer, and a region in which the amount of oxygen gradually increases from an interface between the wiring layer and the second adhesion layer toward the second insulating layer.

[11] The method for manufacturing the microheater according to any one of [7] to [10], further comprising: forming a temperature sensor on the second insulating layer, wherein the temperature sensor comprises: a process of forming a metal oxide layer on the second insulating layer; and a process of forming a metal layer on the metal oxide layer, the second insulating layer comprises: a process of forming an oxide insulating layer on the first insulating layer and the second adhesion layer; and a process of forming a nitride layer on the oxide insulating layer, the wiring layer includes platinum, the wiring layer is formed to include a first bellows structure, the temperature sensor is formed to include a second bellows structure, and an angle formed between a straight line portion of the first bellows structure and a straight line portion of the second bellows structure is in a range from 45 degrees to 135 degrees.

[12] The method for manufacturing the microheater according to [11], wherein a metal oxide in the metal oxide layer includes the oxygen-deficient region in which the oxygen is deficient in a stoichiometric ratio of metal to oxygen.

[13] The method for manufacturing the microheater according to [11], wherein a metal oxide in the metal oxide layer includes a material which is the same as that of the metal oxide in the first adhesion layer and the second adhesion layer.

### FIRST EMBODIMENT

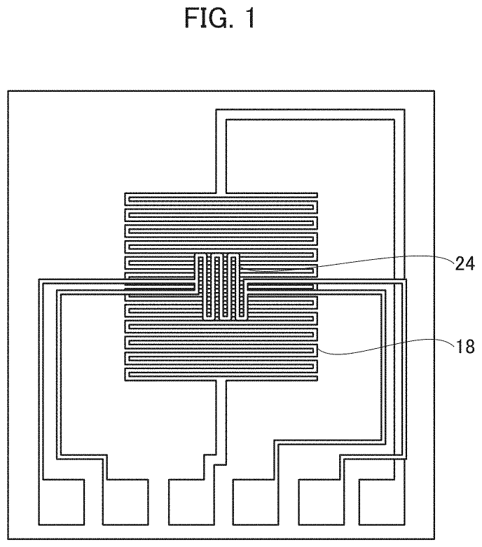

A microheater and a method for manufacturing a microheater according to the present embodiment will be described with reference to FIGS. 1 to 13.

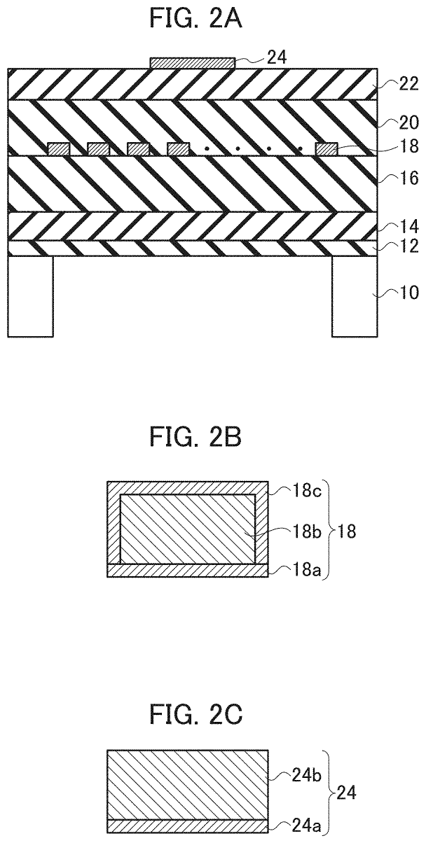

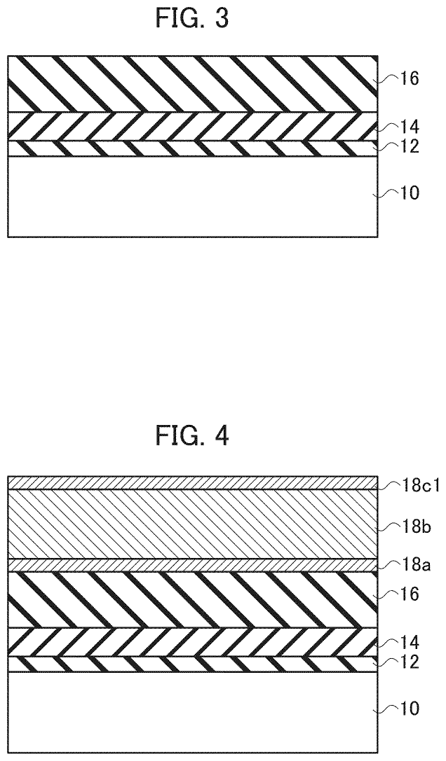

FIG. 1 is a schematic plan view showing a structure of a microheater according to the present embodiment, FIG. 2 is a schematic cross sectional view showing a structure of a microheater according to the present embodiment, and FIGS. 3 to 13 are schematic cross sectional views for explaining a method for manufacturing a microheater according to the present embodiment.

First, a structure of a microheater according to the present embodiment will be described with reference to FIGS. 1 and 2 (2A to 2C).

As shown in FIGS. 1 and 2A, the microheater includes a substrate 10, an insulating layer 12 on the substrate 10, a nitride layer 14, an insulating layer 16, a heater layer 18 on the insulating layer 16, an insulating layer 20 on the heater layer 18, a nitride layer 22, and a temperature sensor 24. Further, as shown in FIG. 2B, the heater layer 18 includes an adhesion layer 18a, a wiring layer 18b, and an adhesion layer 18c. Further, as shown in FIG. 2C, the temperature sensor 24 includes a metal oxide layer 24a and a metal layer 24b. In the present specification, the substrate 10, the insulating layer 12, the temperature sensor 24, and the like are described as part of the microheater. However, the present invention is not limited to this, and it may be interpreted that the substrate 10, the insulating layer 12, the temperature sensor 24, and the like are not part of the microheater.

The heater layer 18 of the microheater according to the present embodiment includes the adhesion layer 18a, the wiring layer 18b, and the adhesion layer 18c. The insulating layer 20 and the insulating layer 16 are disposed above and below the heater layer 18 respectively. The adhesion layer 18a and the adhesion layer 18c function as barrier layers that are provided between the wiring layer 18b, and each of the insulating layer 16 and the insulating layer 20. That is, the wiring layer 18b is completely covered with the adhesion layer 18a and the adhesion layer 18c that function as barrier layers.

A heat source can be generated by causing a current to flow through the wiring layer 18b. The wiring layer 18b can be made of a conductive material, for example, a metal material such as platinum. The wiring portion of a general microheater is usually used at about 300 to 400° C. However, if the part is used in a high temperature region (about 500° C.) at or above the above described temperature range, the deterioration in the microheater is accelerated, and there is a possibility of causing an operation failure of the microheater. The present inventors have solved the above-described problem by adjusting the materials of the adhesion layer 18a, the wiring layer 18b, and the adhesion layer 18c of the heater layer 18 of the microheater. In order to operate a microheater normally for a long period of time in an operating environment of about 500° C., it is necessary to ensure a heat resistance of about 800° C.

As described above, in general, an adhesion layer of a microheater is covered with a nitride layer. If a microheater is used in a high temperature region, the nitride layer and platinum react with each other due to heating, and a void occurs in a wiring portion. The void gradually expands due to being continuously heated, and eventually the wiring portion is disconnected. An oxide is a material which does not react with platinum. However, adhesion between platinum and an oxide is insufficient, and film separation is likely to occur. Therefore, it is difficult to make an oxide insulating layer function as an adhesion layer.

In order to obtain an adhesion layer that suppresses the occurrence of a void in a wiring portion and ensures adhesion with the wiring portion, the adhesion layer 18a and the adhesion layer 18c in the microheater according to the present embodiment each include a metal oxide. The metal oxide includes an oxygen-deficient region in which oxygen is deficient in the stoichiometric ratio of metal to oxygen.

When metal bonds to oxygen, the metal has greater electronegativity than the metal before being bonded to oxygen. Further, platinum has less electronegativity than the metal after being bonded to oxygen. For this reason, the metal after being bonded to oxygen is less likely to bond to platinum than the metal before being bonded to oxygen. In the present embodiment, the amount of oxygen bonded to metal is reduced to be smaller than the stoichiometric ratio. This can further suppress the increase in the electronegativity of the metal after being bonded to oxygen than the case of the stoichiometric composition, and facilitate the bonding of the metal to platinum. Therefore, if a metal oxide contains an oxygen-deficient region, the metal bonds to platinum, and accordingly the adhesion is enhanced.

An oxygen-deficient region is present in the vicinity of the interface between a wiring portion and an adhesion layer. The oxygen-deficient region is present, for example, in a range from the interface to a position 10 to 100 nm distant from the interface, is preferably present in a range from the interface to a position 20 to 80 nm distant from the interface, and is more preferably present in a range from the interface to a position 20 to 50 nm distant from the interface. Further, the metal oxide may also include a region with a stoichiometric composition. The region with the stoichiometric composition is present adjacent to the edge of the oxygen-deficient region on the side away from the interface between the wiring portion and the adhesion layer. Further, the oxygen-deficient region may have a region where the amount of oxygen gradually increases from the interface between the wiring portion and the adhesion layer toward the insulating layer, that is, a region where the composition gets closer to the stoichiometric composition from the interface between the wiring portion and the adhesion layer toward the insulating layer.

The adhesion layer 18a and the adhesion layer 18c contain a metal oxide. The metal in the metal oxide may include, for example, one selected from the group consisting of titanium, chromium, tungsten, molybdenum, and tantalum. The oxygen in the oxygen-deficient region of the metal oxide is preferably 30 to 80%, more preferably 40 to 75%, and still more preferably 45 to 70% of the oxygen of the stoichiometric composition of the metal oxide.

Further, the stoichiometric ratio of metal to oxygen of the metal oxide is more than 1:0.5 and 1:1.5 or less, preferably 1:0.6 or more and 1:1.5 or less, and more preferably 1:0.9 or more and 1:1.4 or less.

The materials of a wiring layer and a metal oxide are not limited to those described above. The materials may be any materials as long as the metal oxide includes an oxygen-deficient region and an increase in the electronegativity of the metal of the metal oxide at the interface between the material of the wiring layer and the material of the metal oxide is suppressed.

Further, the wiring layer 18b is connected to a pair of electrodes disposed outside the microheater which will be described later. The wiring layer 18b has a first bellows structure as shown in FIG. 1 that includes straight line portions and folded portions.

The substrate 10 has a thickness of, for example, about 10 μm, and can be made of silicon, epoxy resin, a ceramic, or the like.

The insulating layer 12 has a thickness of, for example, about 0.1 μm, and can be made of silicon oxide or the like. The insulating layer 12 functions as an etch stop film for processing the substrate 10. The material of the insulating layer 12 is not limited to those described above, and may be any material as long as the material has the function described above.

The nitride layer 14 and the nitride layer 22 may be made of, for example, silicon nitride or the like. The insulating layer 16 and the insulating layer 20 may be made of, for example, silicon oxide. By using silicon nitride and silicon oxide, stress in a membrane composed of a nitride layer and an insulating layer is adjusted.

The temperature sensor 24 includes the metal oxide layer 24a and the metal layer 24b on the metal oxide layer 24a. The metal oxide in the metal oxide layer 24a may include an oxygen-deficient region in which oxygen is deficient in the stoichiometric ratio of metal to oxygen, and can be made of materials similar to those of the adhesion layer 18a and adhesion layer 18c. Further, the metal layer 24b can be made of materials similar to those of the wiring layer 18b. Although not shown, a metal oxide layer containing an oxygen-deficient region may be further provided on the metal layer 24b.

Further, the temperature sensor 24 has a second bellows structure as shown in FIG. 1. The second bellows structure has straight line portions and folded portions. In the present embodiment, the straight line portions of the first bellows structure of the wiring layer 18b and the straight line portions of the second bellows structure of the temperature sensor 24 are configured to be orthogonal to each other, but the present invention is not limited to this. The angle formed between the straight line portions of the first bellows structure of the wiring layer 18b and the straight line portions of the second bellows structure of the temperature sensor 24 is preferably in the range from 45 degrees to 135 degrees. By setting the angle in the above range, the region where the wiring layer 18b and the temperature sensor 24 overlap each other becomes large, and the temperature sensor 24 can sense the temperature of the wiring layer 18b with high sensitivity. From the viewpoint of the area occupied by the microheater, it is more preferable to set the angle to be in the range from 80 degrees to 100 degrees in consideration of electrodes disposed outside the microheater that are connected to the wiring layer 18b and the temperature sensor 24.

A method for manufacturing a microheater according to the present embodiment will now be described with reference to FIGS. 3 to 13.

First, as shown in FIG. 3, the insulating layer 12, the nitride layer 14, and the insulating layer 16 are sequentially formed in this order on the substrate 10. In the present embodiment, a silicon substrate is used as the substrate 10. Silicon oxide which is formed by means of a CVD (chemical vapor deposition) method is used as the material of the insulating layer 12 and the insulating layer 16. Silicon nitride which is formed by means of a CVD method is used as the material of the nitride layer 14.

Next, as shown in FIG. 4, the adhesion layer 18a, the wiring layer 18b, and the adhesion layer 18c1 serving as a part of the adhesion layer 18c, all of which form the heater layer 18, are sequentially formed in this order above the nitride layer 14. In the present embodiment, as the material of the adhesion layer 18a and the adhesion layer 18c1, titanium oxide which has an oxygen-deficient region and is formed by means of a sputtering method is used, specifically, titanium oxide in which the stoichiometric ratio of titanium to oxygen is about 1:1.1 is used. Platinum which is formed by means of a sputtering method is used as the material of the wiring layer 18b.

In the present embodiment, by using a metal oxide having an oxygen-deficient region as the material of an adhesion layer of the heater layer 18, it is possible to suppress an increase in the electronegativity of the metal of the metal oxide at the interface between the material of a wiring layer and the material of the metal oxide. Accordingly, while suppressing the occurrence of a void in a wiring portion, film separation is also suppressed and the adhesion between the adhesion layer and the wiring portion can be ensured.

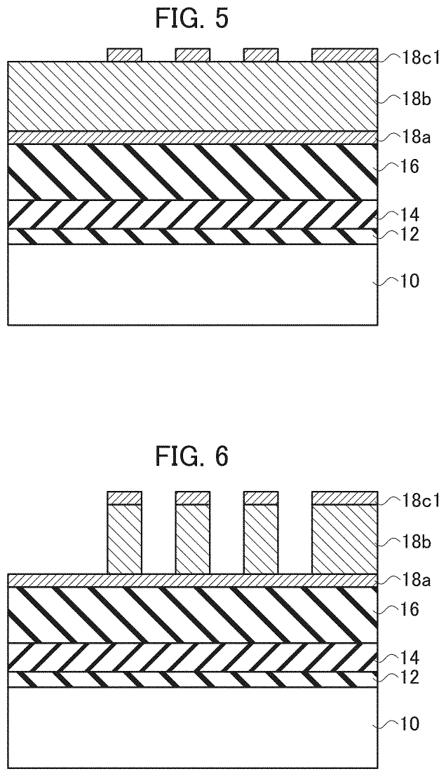

Next, as shown in FIG. 5, the adhesion layer 18c1 is formed. In the step of forming the adhesion layer 18c1, first, a resist is pattern-formed on the adhesion layer 18c1 by means of photolithography. The adhesion layer 18c1 shown in FIG. 5 is formed by removing a part of the adhesion layer 18c1 by using the pattern-formed resist.

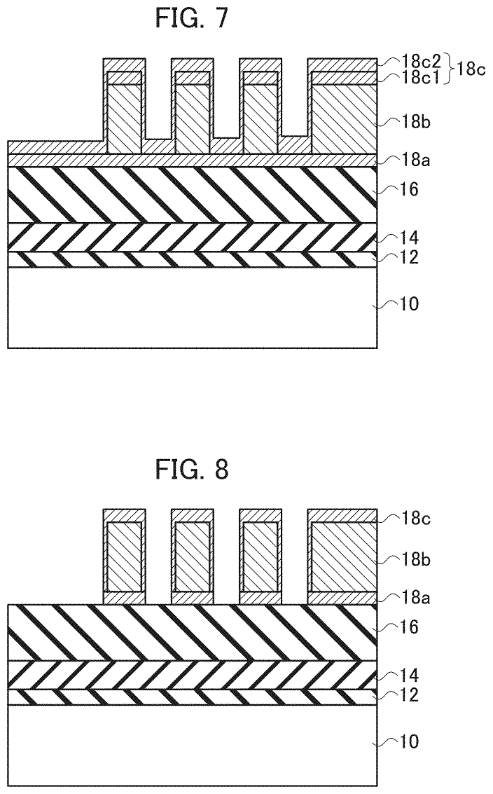

Next, as shown in FIG. 6, by using the pattern-formed resist and the adhesion layer 18c1, a part of the wiring layer 18b is removed and the resist is removed. Accordingly, the wiring layer 18b shown in FIG. 6 is formed. However, the timing for removing the resist is not limited to this. If, for example, pattern-forming of the wiring layer 18b is possible only with the adhesion layer 18c1, the resist may be removed after removing a part of the adhesion layer 18c1. Next, as shown in FIG. 7, an adhesion layer 18c2 which is a part of the adhesion layer 18c is formed above the adhesion layer 18a and on the adhesion layer 18c1. The combination of the adhesion layer 18c1 and the adhesion layer 18c2 corresponds to the adhesion layer 18c. The material of the adhesion layer 18c2 may be the similar to that of the adhesion layer 18c1 described above.

Next, as shown in FIG. 8, the adhesion layer 18c is formed. The wiring layer 18b is configured to be completely covered with the adhesion layer 18a and the adhesion layer 18c. In the step of forming the adhesion layer 18c1, first, a resist is pattern-formed on the adhesion layer 18c by means of photolithography. By removing a part of the adhesion layer 18a and a part of the adhesion layer 18c by using the pattern-formed resist, the adhesion layer 18a and the adhesion layer 18c shown in FIG. 8 are formed. In the present embodiment, the adhesion layer 18c has a two-layer structure of the adhesion layer 18c1 and the adhesion layer 18c2, but the structure is not limited to this, and the adhesion layer 18c may only have the adhesion layer 18c2 without providing the adhesion layer 18c1.

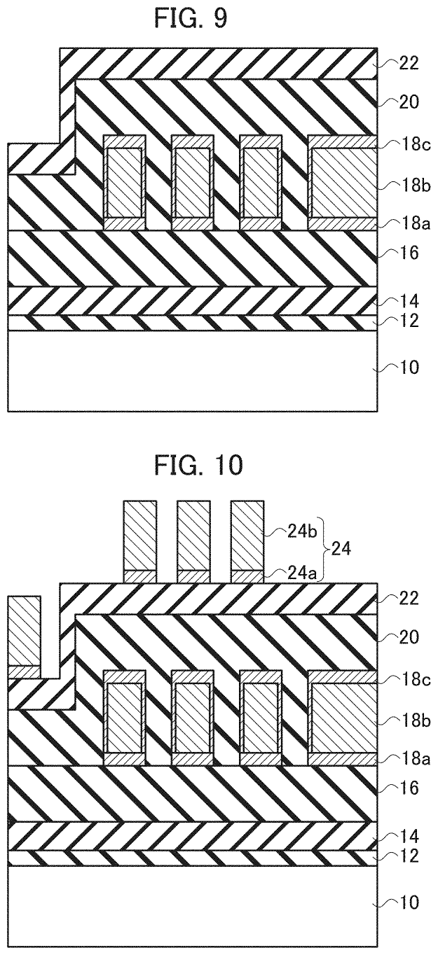

Next, as shown in FIG. 9, the insulating layer 20 and the nitride layer 22 are sequentially formed in this order above the insulating layer 16 and the heater layer 18. In the present embodiment, silicon oxide formed by means of a CVD method is used as the material of the insulating layer 20. Silicon nitride formed by means of a CVD method is used as the material of the nitride layer 22.

Next, as shown in FIG. 10, the temperature sensor 24 including the metal oxide layer 24a and the metal layer 24b is formed on the nitride layer 22. In the present embodiment, as the material of the metal oxide layer 24a, titanium oxide which has an oxygen-deficient region and is formed by means of a sputtering method is used, specifically, titanium oxide in which the stoichiometric ratio of titanium to oxygen is about 1:1.1 is used. Platinum formed by means of a sputtering method is used as the material of the metal layer 24b.

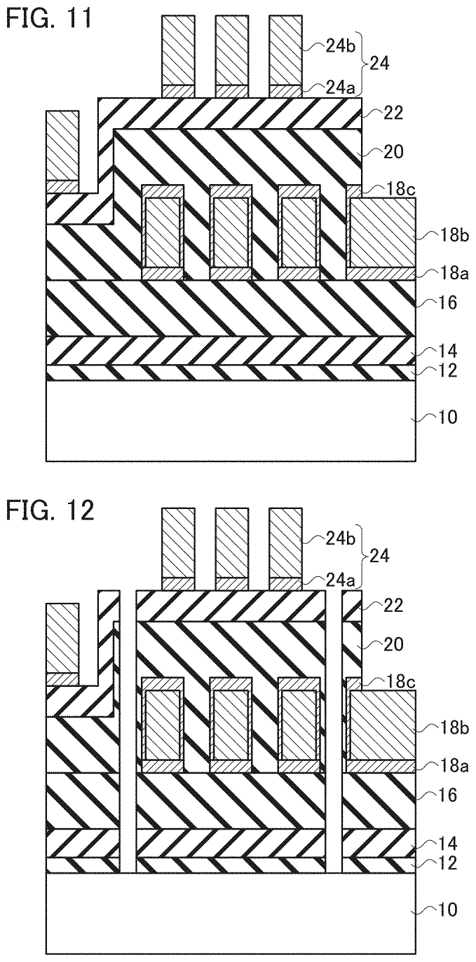

Next, as shown in FIG. 11, openings for connecting a pair of electrodes disposed outside the microheater and the wiring layer 18b are formed. In the step of forming the openings, first, a resist is pattern-formed on the nitride layer 22 and the temperature sensor 24 by means of photolithography. The openings shown in FIG. 11 are formed by removing a part of the nitride layer 22, a part of the insulating layer 20, and a part of the adhesion layer 18c by using the pattern-formed resist.

Next, as shown in FIG. 12, openings which extend to reach the substrate 10 are formed. In the step of forming the openings, as described above, a resist is pattern-formed by means of photolithography, and the openings are formed by using the pattern-formed resist.

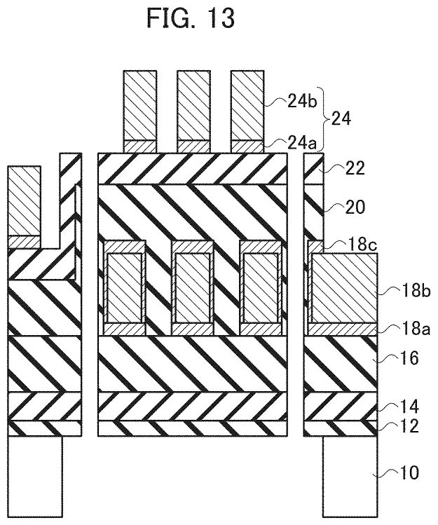

Finally, as shown in FIG. 13, the microheater according to the present embodiment can be manufactured by removing a part of the substrate 10 by means of etching or the like.

According to the present embodiment, it is possible to provide a microheater with an adhesion layer that suppresses the occurrence of a void in a wiring portion even in a high temperature region and ensures adhesion with the wiring portion. This can suppress operation failures of a microheater and can ensure reliability.

### SECOND EMBODIMENT

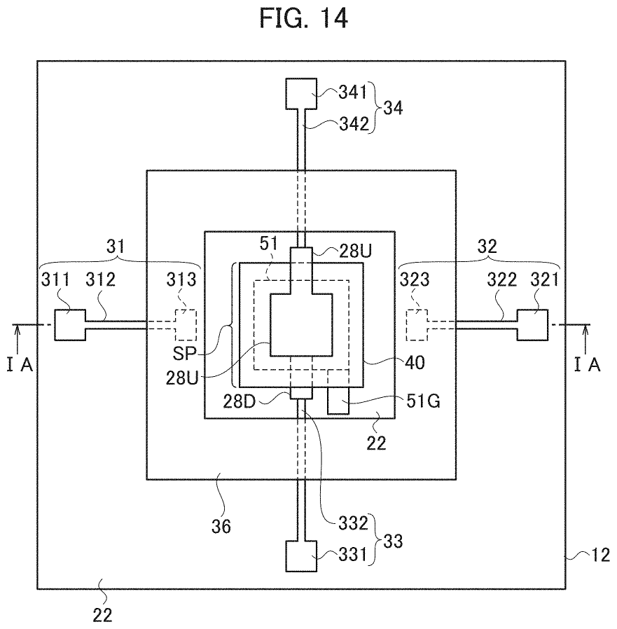

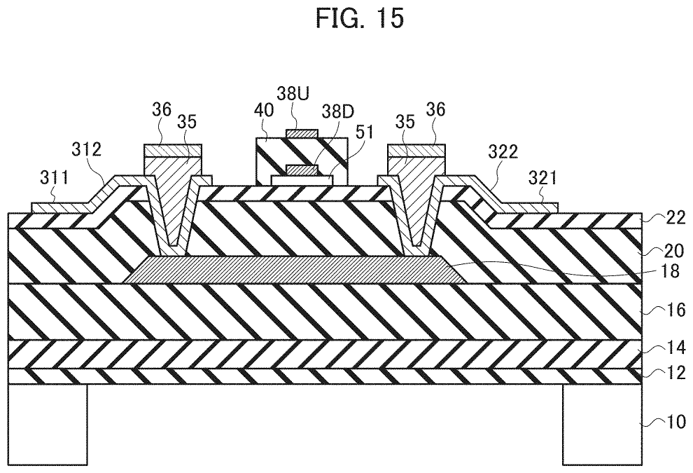

A gas sensor having a microheater according to the first embodiment will be described with reference to FIGS. 14 and 15.

FIG. 14 is a schematic plan view of a gas sensor having a microheater, and FIG. 15 is a schematic cross sectional view of the gas sensor which is taken along line IA-IA of FIG. 14.

As shown in FIGS. 14 and 15, the gas sensor includes a microheater having a temperature sensor provided on the substrate 10, a heater connection unit 31, a heater connection unit 32, a terminal electrode connection unit 33, a terminal electrode connection unit 34, and the like. The microheater described in the first embodiment can be used as the microheater.

A sensor part SP having a temperature sensor includes a porous oxide film (a porous film) 51 that is disposed via the nitride layer 22, a lower electrode 38D disposed on the porous oxide film 51, a solid electrolyte layer 40 disposed so as to cover the porous oxide film 51 and the lower electrode 38D, and an upper electrode 38U that is disposed on the solid electrolyte layer 40 and faces the lower electrode 38D. The porous oxide film 51 functions as a gas introduction path and has a gas intake port 51G.

The temperature sensor described in the first embodiment can be used for the lower electrode 38D and the upper electrode 38U.

The solid electrolyte layer 40 may be formed of a YSZ film having a thickness of about 1 μm. This is because, if the layer is thin, the lower electrode 38D and the upper electrode 38U are electrically connected. For example, the solid electrolyte layer 40 is disposed so as to cover the periphery of the lower electrode 38D, and can prevent conduction between the lower electrode 38D and the upper electrode 38U.

In plan view, the porous oxide film 51, the lower electrode 38D, the solid electrolyte layer 40, and the upper electrode 38U of the sensor part SP may all have a square shape, or may have other shapes. The porous oxide film 51, the lower electrode 38D, the solid electrolyte layer 40, and the upper electrode 38U forming the sensor part SP are desirably disposed in the center of the sensor surface without eccentricity. However, they may be disposed in an eccentric state if they are disposed on the microheater.

In plan view, the heater connection unit 31 and the heater connection unit 32 are disposed so as to face each other across the sensor part SP in the left-right direction in the drawing (in-plane direction along the cross section of FIG. 15). The heater connection unit 31 has a connection pad 311, a wiring portion 312, and a terminal portion 313. The heater connection unit 32 has a connection pad 321, a wiring portion 322, and a terminal portion 323. The terminal electrode connection unit 33 and the terminal electrode connection unit 34 are disposed so as to face each other across the sensor part SP in a direction orthogonal to the heater connection unit 31 and the heater connection unit 32, which is the vertical direction in the drawing. The terminal electrode connection unit 33 has a connection pad (a detection terminal) 331 and a wiring portion 332. The terminal electrode connection unit 34 has a connection pad (a detection terminal) 341 and a wiring portion 342. The heater connection units and the terminal electrode connection units described above can use the structure of the adhesion layer and the wiring layer described in the first embodiment.

The heater connection unit 31, the heater connection unit 32, the terminal electrode connection unit 33, and the terminal electrode connection unit 34 are disposed on the nitride layer 22.

The terminal portion 313 and the terminal portion 323 of the heater connection unit 31 and the heater connection unit 32 are connected to the microheater. The wiring portion 332 of the terminal electrode connection unit 33 is connected to the lower electrode 38D that extends in the direction of the sensor part SP. The wiring portion 342 of the terminal electrode connection unit 34 is connected to the upper electrode 38U that extends in the direction of the sensor part SP.

The terminal portion 313 and the terminal portion 323 of the heater connection unit 31 and the heater connection unit 32 are covered with a silicon nitride layer 36. The silicon nitride layer 36 is disposed so as to surround the outer peripheral part of the sensor part SP in plan view. A silicon oxide layer 35 is embedded between the silicon nitride layer 36 and each of the terminal portions 313 and 323.

A detection circuit for detecting a predetermined gas concentration in a gas to be measured is connected to the connection pad 331 and the connection pad 341 of the terminal electrode connection unit 33 and the terminal electrode connection unit 34. A detection voltage V is supplied to the upper electrode 38U and a porous electrode 51 of the solid electrolyte layer 40. Accordingly, the detection circuit can detect the oxygen concentration based on the limiting current. Further, the detection circuit can detect the water vapor concentration based on the limiting current.

The gas sensor according to the present embodiment is configured to introduce a gas to be measured (for example, O₂ gas) into the solid electrolyte layer 40 of the sensor part SP through the gas intake port 51G of the porous oxide film 51 in accordance with heating of the microheater. That is, the gas to be measured is taken into the porous oxide film 51 through the gas intake port 51G, and is introduced into the solid electrolyte layer 40 through the lower electrode 38D. Thereafter, the gas to be measured is diffused into the solid electrolyte layer 40 due to the heating. The introduction of the gas to be measured into the solid electrolyte layer 40 may also include a suction operation.

Furthermore, the gas sensor according to the present embodiment includes the microheater according to the first embodiment. It is possible to suppress operation failures of the microheater because the occurrence of a void in a wiring portion of the microheater is suppressed even in a high temperature region, and an adhesion layer of the microheater has good adhesion with the wiring portion. Accordingly, the gas sensor according to the present embodiment can suppress operation failures, and ensure reliability.

### OTHER EMBODIMENTS

As described above, although several embodiments have been described, it should be understood that the discussion and drawings that form part of the disclosure are illustrative and not limiting. Various alternative embodiments, examples, and operating techniques will become apparent to those skilled in the art from this disclosure. For example, the configuration according to the present embodiment can be applied to sensors such as a flow sensor and a carbon dioxide detection sensor. In this way, the present embodiment includes various embodiments and the like, such as combinations of each embodiment and each example, which are not described herein.

### EXAMPLES

Although the above embodiments will be described in more detail below with reference to examples, the above embodiments are not limited to the following examples.

### Example 1

In the present example, a cross section TEM in the microheater described above was observed.

As shown in the first embodiment, the microheater of the present example includes a silicon substrate which is a substrate 10, an insulating layer 12, a nitride layer 14, an insulating layer 16, a heater layer 18, an insulating layer 20, and a nitride layer 22. An insulating layer 25 is formed on the nitride layer 22.

The insulating layer 12 is a silicon oxide layer, the nitride layer 14 is a silicon nitride layer, the insulating layer 16 is a silicon oxide layer, the insulating layer 20 is a silicon oxide layer, the nitride layer 22 is a silicon nitride layer, and the insulating layer 25 is a silicon oxide layer. These insulating layers were formed by means of a CVD method.

The heater layer 18 includes an adhesion layer 18a, a wiring layer 18b, and an adhesion layer 18c. The adhesion layer 18a is a titanium oxide layer in which the stoichiometric ratio of titanium to oxygen is 1:1.1, and the wiring layer 18b is a platinum layer. The adhesion layer 18c is a titanium oxide layer in which the stoichiometric ratio of titanium to oxygen is 1:1.1. The adhesion layer 18a, the wiring layer 18b, and the adhesion layer 18c were formed by means of a sputtering method.

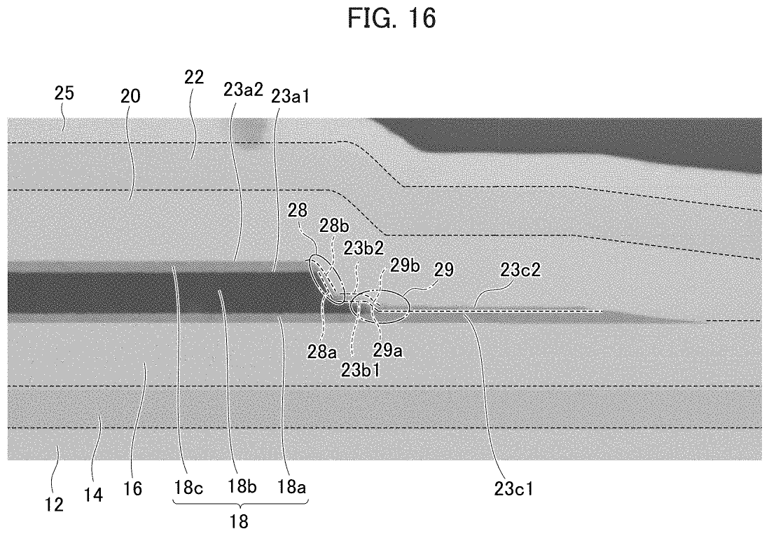

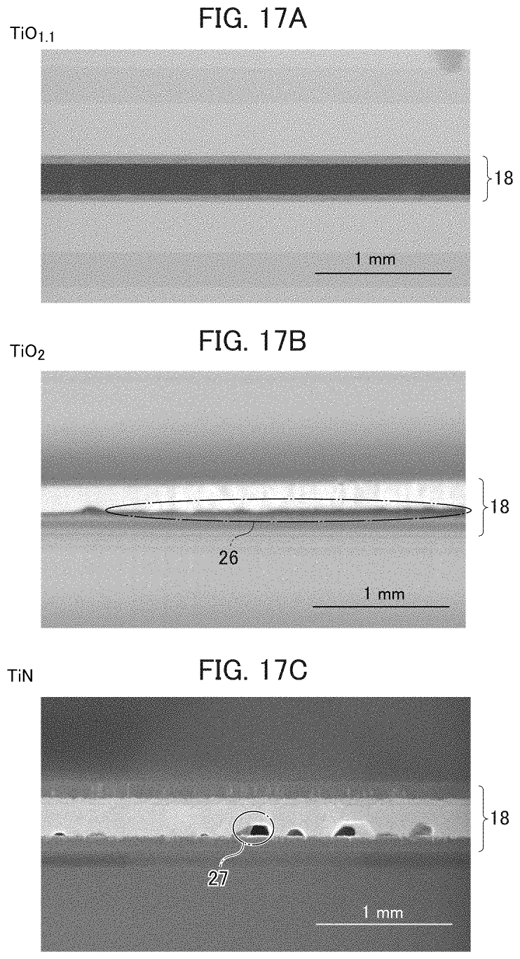

After a heat treatment was performed at 700° C. in the microheater, a cross section TEM was observed. A field-emission transmission electron microscope JEM-2800 manufactured by Hitachi High-Tech Corporation was used for the observation of the cross section TEM. FIG. 16 shows an obtained image of the cross section TEM. In FIG. 16, auxiliary lines are added to indicate boundaries between layers. As shown in FIG. 16, the wiring layer 18b has an upper end portion 28 and a lower end portion 29. The lower end portion 29 projects further than the upper end portion 28. This structure is formed when the wiring layer 18b is etched by using a resist pattern as a mask in which etching was performed so that a side surface of the wiring layer 18b is inclined. The covering property of the adhesion layer 18c on the wiring layer 18b is enhanced by the shape of the wiring layer 18b. Further, the distance between a lower surface 28a and an upper surface 28b of the adhesion layer 18c at the upper end portion 28 (the thickness of the adhesion layer 18c at the upper end portion 28) and the distance between a lower surface 29a and an upper surface 29b of the adhesion layer 18c at the lower end portion 29 (the thickness of the adhesion layer 18c at the lower end portion 29) are smaller than the thickness of the adhesion layer 18c at portions other than the upper end portion 28 and the lower end portion 29 due to the difference in the covering property between the upper surface portion and the side surface portion of the adhesion layer 18c. Examples of the thickness of the adhesion layer 18c at portions other than the upper end portion 28 and the lower end portion 29 include the distance between a lower surface 23a1 and an upper surface 23a2 of the adhesion layer 18c, the distance between a lower surface 23b1 and an upper surface 23b2, and the distance between a lower surface 23c1 and an upper surface 23c2 (the thickness of the adhesion layer 18c at portions other than the upper end portion 28 and the lower end portion 29). FIG. 17A shows an enlarged view of the vicinity of the heater layer 18 in FIG. 16.

As shown in FIG. 17A, the occurrence of a void in the wiring layer 18b was not found, and film separation at the interface between the wiring layer 18b and the adhesion layer 18a or 18c was not found either.

### Example 2

In the present example, whether a void and film separation occur due to the difference in the material of an adhesion layer in the microheater of Example 1 was evaluated by means of cross section TEM observation and surface microscope observation.

As evaluation samples for the cross section TEM observation, three kinds of layers were prepared for adhesion layers 18a and 18c. The three kinds of layers were a titanium oxide layer (TiO_{1.1}) in which the stoichiometric ratio of titanium to oxygen is 1:1.1, a titanium oxide layer (TiO₂) in which the stoichiometric ratio of titanium to oxygen is 1:2 (a stoichiometric composition), and a titanium nitride layer (TiN) in which the stoichiometric ratio of titanium to nitrogen is 1:1. The ratio of oxygen in the titanium oxide layer (TiO_{1.1}) is 55% of oxygen in the case of the stoichiometric composition of titanium oxide (hereinafter the ratio of oxygen in TiOₓ to TiO₂ is also referred to as the oxygen ratio). The titanium nitride layer (TiN) was prepared as a sample of a layer not containing oxygen. The prepared samples were heat-treated at 700° C.

As evaluation samples of surface micrographs, six kinds of layers were prepared for the adhesion layers 18a and 18c. The six kinds of layers were a titanium oxide layer (TiO_{0.5}: oxygen ratio 25%), a titanium oxide layer (TiO_{0.9}: oxygen ratio 45%), a titanium oxide layer (TiO_{1.1}: oxygen ratio 55%), a titanium oxide layer (TiO_{1.4}: oxygen ratio 70%), a titanium oxide layer (TiO₂: oxygen ratio 100%), and a titanium nitride layer (TiN: oxygen ratio 0%). The prepared samples were heat-treated at 800° C.

FIGS. 17A to 17C show cross section TEM images which were obtained in the same manner as in Example 1. FIG. 17A shows a cross section where the adhesion layer 18a and the adhesion layer 18c are titanium oxide layers (TiO_{1.1}). FIG. 17B shows a cross section where the adhesion layer 18a and the adhesion layer 18c are titanium oxide layers (TiO₂). FIG. 17C shows a cross section where the adhesion layer 18a and the adhesion layer 18c are titanium nitride layers (TiN).

As shown in FIG. 17A, when the adhesion layer 18a and the adhesion layer 18c are titanium oxide layers (TiO_{1.1}), the occurrence of a void in the wiring layer 18b was not found, and the occurrence of film separation at the interface between the wiring layer 18b, and the adhesion layer 18a or 18c was not found either. Meanwhile, as shown in FIG. 17B, when the adhesion layer 18a and the adhesion layer 18c are titanium oxide layers (TiO₂), the occurrence of film separation in the region 26 was found. Further, as shown in FIG. 17C, when the adhesion layer 18a and the adhesion layer 18c are titanium nitride layers (TiN), the occurrence of a void in the region 27 was found.

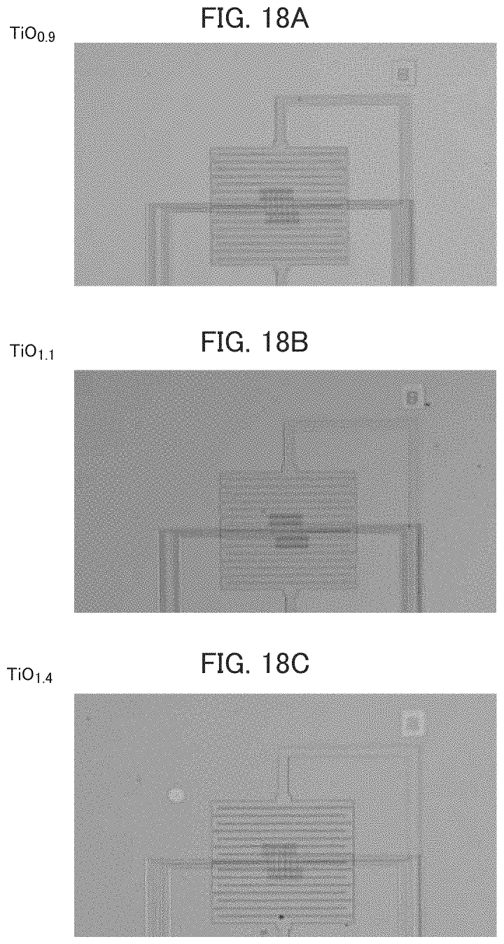

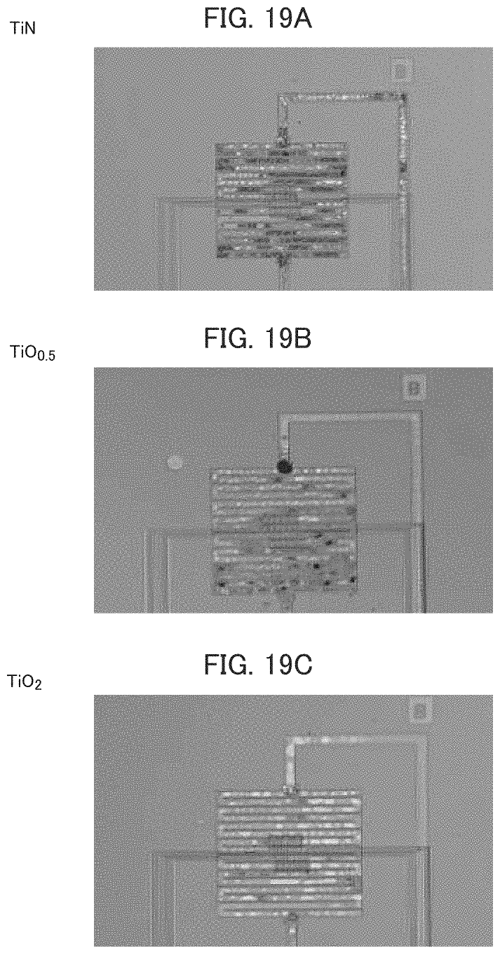

FIGS. 18A to 18C and 19A to 19C show obtained surface micrographs. FIG. 18A shows a surface where the adhesion layer 18a and the adhesion layer 18c are titanium oxide layers (TiO_{0.9}). FIG. 18B shows a surface where the adhesion layer 18a and the adhesion layer 18c are titanium oxide layers (TiO_{1.1}). FIG. 18C shows a surface where the adhesion layer 18a and the adhesion layer 18c are titanium oxide layers (TiO_{1.4}). FIG. 19A shows a surface where the adhesion layer 18a and the adhesion layer 18c are titanium nitride layers (TiN). FIG. 19B shows a surface where the adhesion layer 18a and the adhesion layer 18c are titanium oxide layers (TiO_{0.5}). FIG. 19C shows a surface where the adhesion layer 18a and the adhesion layer 18c are titanium oxide layers (TiO₂).

As shown in FIGS. 18A to 18C, when the materials of the adhesion layer 18a and the adhesion layer 18c are the titanium oxide layer (TiO_{0.9}), the titanium oxide layer (TiO_{1.1}), and the titanium oxide layer (TiO_{1.4}), the occurrence of a void was not found, and the occurrence of film separation at the interface between the wiring layer 18b, and the adhesion layer 18a or the adhesion layer 18c was not found either. Meanwhile, as shown in FIGS. 19A to 19C, when the materials of the adhesion layer 18a and the adhesion layer 18c are the titanium nitride layer (TiN), the titanium oxide layer (TiO_{0.5}), and the titanium oxide layer (TiO₂), the occurrence of a void or film separation (the black points and white points in the drawings) was found.

From the above evaluation results, it was found that the use of a titanium oxide layer in which the stoichiometric ratio of titanium to oxygen is 1:0.9 to 1.4 was preferable.

### Example 3

In the present example, the performance of a microheater in which the adhesion layer 18a and the adhesion layer 18c used in Example 1 are titanium oxide layers (TiO_{1.1}) was evaluated.

First, the temperature of a membrane that includes a nitride layer and an insulating layer, and the electric power applied to the wiring layer 18b were evaluated.

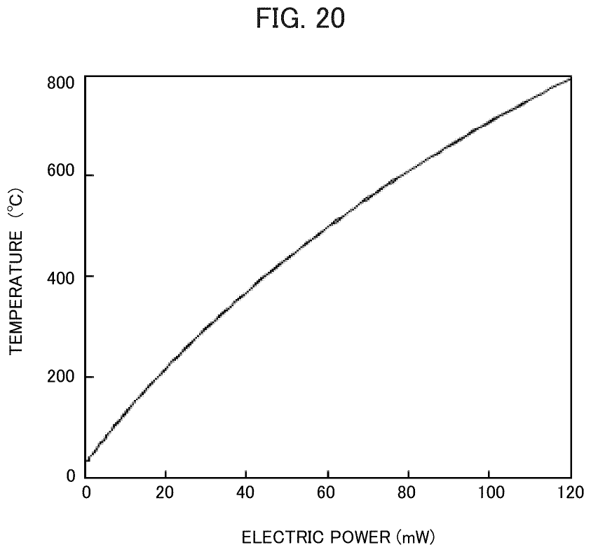

FIG. 20 shows the evaluation results. As shown in FIG. 20, it was found that, if electric power of 120 mW is applied to the wiring layer 18b, the temperature of the membrane reaches 800° C. Before and after the temperature of the membrane reaches 800° C., there is no change in resistance and no hysteresis was observed. Therefore, it was found that the microheater did not deteriorate.

Next, the cycle characteristic (current change) of the microheater was evaluated by repeated testing of the microheater at 550° C. and room temperature (25° C.), at a cycle of 0.2 seconds and a duty ratio of 50%. The microheater was connected in parallel with three elements, and the voltage was fixed at 8 V.

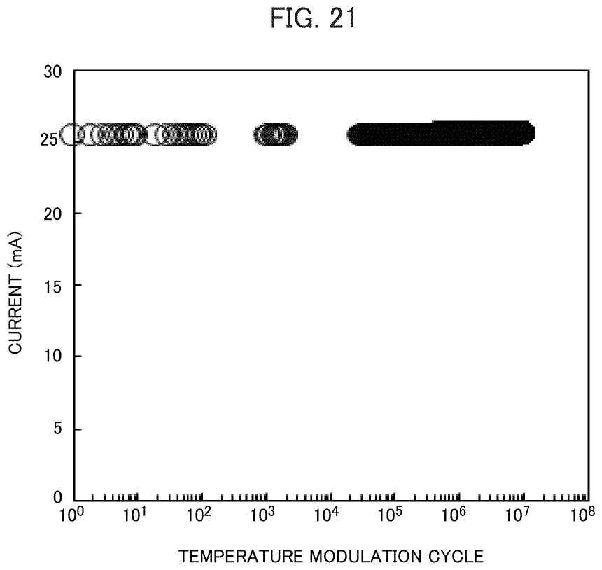

FIG. 21 shows the evaluation results. As shown in FIG. 21, even if the test was repeated 10⁷ times, the current of the microheater did not change, and the resistance of the microheater did not change. That is, it was found that the microheater did not deteriorate.

### REFERENCE SIGNS LIST

10 . . . Substrate, 12 . . . Insulating layer, 14 . . . Nitride layer, 16 . . . Insulating layer, 18 . . . Heater layer, 18a . . . Adhesion layer, 18b . . . Wiring layer, 18c . . . Adhesion layer, 18c1 . . . Adhesion layer, 18c2 . . . Adhesion layer, 20 . . . Insulating layer, 22 . . . Nitride layer, 23a1 . . . Lower surface, 23a2 . . . Upper surface, 23b1 . . . Lower surface, 23b2 . . . Upper surface, 23c1 . . . Lower surface, 23c2 . . . Upper surface, 24 . . . Temperature sensor, 24a . . . Metal oxide layer, 24b . . . Metal layer, 25 . . . Insulating layer, 26 . . . Region, 27 . . . Region, 28 . . . Upper end portion, 28a . . . Lower surface, 28b . . . Upper surface, 29 . . . lower end portion, 29a . . . Lower surface, 29b . . . Upper surface, 31 . . . Heater connection unit, 32 . . . Heater connection unit, 33 . . . Terminal electrode connection unit, 34 . . . Terminal electrode connection unit, 35 . . . Silicon oxide layer, 36 . . . Silicon nitride layer, 38D . . . Lower electrode, 38U . . . Upper electrode, 40 . . . Solid electrolyte layer, 51 . . . Porous oxide film, 51G . . . Gas intake port, 311 . . . Connection pad, 312 . . . Wiring portion, 313 . . . Terminal portion, 321 . . . Connection pad, 322 . . . Wiring portion, 323 . . . Terminal portion, 331 . . . Connection pad, 332 . . . Wiring portion, 341 . . . Connection pad, 342 . . . Wiring portion

What is claimed is:

1. A microheater comprising:
a first insulating layer;
a first adhesion layer on the first insulating layer;
a wiring layer on the first adhesion layer;
a second adhesion layer that covers the wiring layer; and
a second insulating layer above the first insulating layer and on the second adhesion layer, wherein
the wiring layer includes platinum,
the first adhesion layer and the second adhesion layer each include a metal oxide, and
the metal oxide includes an oxygen-deficient region in which oxygen is deficient in a stoichiometric ratio of metal to oxygen.

2. The microheater according to claim 1, wherein
the oxygen in the oxygen-deficient region is 30 to 80% of oxygen of a stoichiometric composition of the metal oxide, and
the metal includes one selected from the group consisting of titanium, chromium, tungsten, molybdenum, and tantalum.

3. The microheater according to claim 1, wherein
the metal is titanium, and
a stoichiometric ratio of metal to oxygen of the metal oxide is more than 1:0.5 and 1:1.5 or less.

4. The microheater according to claim 1, wherein
the oxygen-deficient region includes a region in which the amount of oxygen gradually increases from an interface between the wiring layer and the first adhesion layer toward the first insulating layer, and a region in which the amount of oxygen gradually increases from an interface between the wiring layer and the second adhesion layer toward the second insulating layer.

5. The microheater according to claim 1, further comprising:
a temperature sensor on the second insulating layer, wherein
the wiring layer includes platinum,
the second insulating layer includes an oxide insulating layer and a nitride layer on the oxide insulating layer,
the wiring layer is connected to each of a pair of electrodes and includes a first bellows structure,
the temperature sensor includes a second bellows structure,
an angle formed between a straight line portion of the first bellows structure and a straight line portion of the second bellows structure is in a range from 45 degrees to 135 degrees, and
the temperature sensor includes a metal oxide layer and a metal layer on the metal oxide layer.

6. A gas sensor that includes the microheater according to claim 5, wherein
a metal oxide in the metal oxide layer includes the oxygen-deficient region in which the oxygen is deficient in a stoichiometric ratio of metal to oxygen, and
the metal oxide in the metal oxide layer includes a material which is the same as that of the metal oxide in the first adhesion layer and the second adhesion layer.

7. A method for manufacturing a microheater comprising:
forming a first insulating layer;
forming a first adhesion layer on the first insulating layer;
forming a wiring layer on the first adhesion layer;
forming a second adhesion layer that covers a side surface of the wiring layer on the wiring layer; and
forming a second insulating layer above the first insulating layer and on the second adhesion layer, wherein
the first adhesion layer and the second adhesion layer each include a metal oxide, and
the metal oxide includes an oxygen-deficient region in which oxygen is deficient in a stoichiometric ratio of metal to oxygen.

8. The method for manufacturing the microheater according to claim 7, wherein
the oxygen in the oxygen-deficient region is 30 to 80% of oxygen of a stoichiometric composition of the metal oxide, and
the metal includes one selected from the group consisting of titanium, chromium, tungsten, molybdenum, and tantalum.

9. The method for manufacturing the microheater according to claim 7, wherein
the metal is titanium, and
a stoichiometric ratio of metal to oxygen of the metal oxide is more than 1:0.5 and 1:1.5 or less.

10. The method for manufacturing the microheater according to claim 7, wherein
the oxygen-deficient region includes a region in which the amount of oxygen gradually increases from an interface between the wiring layer and the first adhesion layer toward the first insulating layer, and a region in which the amount of oxygen gradually increases from an interface between the wiring layer and the second adhesion layer toward the second insulating layer.

11. The method for manufacturing the microheater according to claim 7, further comprising:
forming a temperature sensor on the second insulating layer, wherein
the temperature sensor comprises:
a process of forming a metal oxide layer on the second insulating layer; and
a process of forming a metal layer on the metal oxide layer,
the second insulating layer comprises:
a process of forming an oxide insulating layer on the first insulating layer and the second adhesion layer; and
a process of forming a nitride layer on the oxide insulating layer,
the wiring layer includes platinum,
the wiring layer is formed to include a first bellows structure,
the temperature sensor is formed to include a second bellows structure, and
an angle formed between a straight line portion of the first bellows structure and a straight line portion of the second bellows structure is in a range from 45 degrees to 135 degrees.

12. The method for manufacturing the microheater according to claim 11, wherein
a metal oxide in the metal oxide layer includes the oxygen-deficient region in which the oxygen is deficient in a stoichiometric ratio of metal to oxygen.

13. The method for manufacturing the microheater according to claim 11, wherein
a metal oxide in the metal oxide layer includes a material which is the same as that of the metal oxide in the first adhesion layer and the second adhesion layer.
